(19) United States
(12) Patent Application Publication
(10) Pub. No.: US 2026/0263149 A1
(43) Pub. Date: Sep. 10, 2026

(54) ELECTROCAUTERY DEVICES, SYSTEMS AND METHODS FOR TRANSSEPTAL ACCESS

(71) Applicant: The Nemours Foundation, Jacksonville, FL (US)

(72) Inventors: Christopher M. Simoni, Saint Cloud, FL (US); Nathan Kostick, Kissimmee, FL (US); Ali Seyfi Yalim Yalcin, Orlando, FL (US)

(21) Appl. No.: 19/557,404

(22) Filed: Mar. 5, 2026

Related U.S. Application Data
(60) Provisional application No. 63/767,685, filed on Mar. 6, 2025.

(51) Int. Cl. A61B 18/14; A61B 18/00

(52) U.S. Cl. CPC A61B 18/1492; A61B 2018/00095; A61B 2018/00101; A61B 2018/00351; A61B 2018/00595; A61B 2018/1435

(57) ABSTRACT

An electrocautery device for transseptal access to a cardiac chamber of a patient. The device includes a proximal end segment, an intermediate segment, and a distal end segment. The proximal end segment is configured to remain external from the patient. The intermediate segment is coupled to the proximal end segment, the intermediate segment configured to be at least partially disposed in the heart of the patient, and a portion of the intermediate segment is reconfigurable from an axially elongated configuration to an axially shortened configuration. The distal end segment is coupled to the intermediate segment opposite the proximal end segment. The distal end segment is configured to be disposed in the heart of the patient, and the distal end segment is configured to deliver thermal energy to a cardiac septum of the heart of the patient and form a perforation through the cardiac septum.

### CROSS-REFERENCE TO RELATED APPLICATION

The present application claims the benefit of and priority to, under 35 U.S.C. §119(e), U.S. Provisional Application Ser. No. 63/767,685, filed on Mar. 6, 2025, which is hereby incorporated by reference in its entirety for all purposes.

### FIELD OF THE DISCLOSURE

The present disclosure generally relates to medical devices for transseptal access to a cardiac chamber of a patient. More specifically, the present disclosure relates to electrocautery devices for transseptal access to the left atrium of a patient.

### BACKGROUND OF THE DISCLOSURE

Transseptal access to the left atrium is utilized in various types of procedures, including transcatheter mitral valve repair (TMVr), mitral valve in valve (VinV) implantation, transcatheter mitral valve replacement (TMVR), creating pressure-reducing shunts for atrial decompression, and the like. Such access is typically provided by using a stiff needle to puncture the atrial septum. However, such a procedure introduces the risk of cardiac perforation. In other cases, transseptal access is provided by using radiofrequency (RF) energy delivery devices. However, RF energy delivery is relatively diffuse making it difficult to pass through the tissue and has limited effectiveness when used for puncturing tissue. As a result, multiple attempts may be needed to cross the atrial septum, which increases procedure time and thereby increases patient risk from anesthesia and radiation exposure. Accordingly, improved transseptal access devices, systems, and methods would be beneficial.

### SUMMARY OF THE INVENTION

In one aspect, the present disclosure provides an electrocautery device for transseptal access to a cardiac chamber of a patient. The device includes a proximal end segment, an intermediate segment, and a distal end segment. The proximal end segment is configured to remain external from the patient. The intermediate segment is coupled to the proximal end segment, the intermediate segment configured to be at least partially disposed in the heart of the patient, and a portion of the intermediate segment is reconfigurable from an axially elongated configuration to an axially shortened configuration. The distal end segment is coupled to the intermediate segment opposite the proximal end segment. The distal end segment is configured to be disposed in the heart of the patient, and the distal end segment is configured to deliver thermal energy to a cardiac septum of the heart of the patient and form a perforation through the cardiac septum.

In another aspect, the present disclosure provides an electrocautery system for transseptal access to a cardiac chamber of a patient. The system includes an energy source configured to provide electrical energy. An electrocautery device is configured to receive the electrical energy from the energy source and convert the electrical energy to thermal energy. The electrocautery device includes a proximal end segment configured to remain external from the patient. The electrocautery device further includes an intermediate segment coupled to the proximal end segment. The intermediate segment is configured to be at least partially disposed in the heart of the patient, and a portion of the intermediate segment is reconfigurable from an axially elongated configuration to an axially shortened configuration. The electrocautery device further includes a distal end segment coupled to the intermediate segment opposite the proximal end segment. The distal end segment is configured to be disposed in the heart of the patient, and the distal end segment is configured to deliver the thermal energy to a cardiac septum of the heart of the patient and form a perforation through the cardiac septum.

In another aspect, the present disclosure provides a method for transseptal access to a cardiac chamber of a patient. The method includes the steps of: advancing a distal end segment of an electrocautery device from an access site on the patient to a first side of a cardiac septum in the heart of the patient; delivering thermal energy from the distal end segment of the electrocautery device to the cardiac septum; advancing the distal end segment and a portion of an intermediate segment of the electrocautery device, in an axially elongated configuration, from the first side of the cardiac septum to a second side of the cardiac septum, the electrocautery device thereby forming a perforation through the cardiac septum; and reconfiguring the portion of the intermediate segment of the electrocautery device from the axially elongated configuration to an axially shortened configuration on the second side of the cardiac septum.

### BRIEF DESCRIPTION OF THE DRAWINGS

For illustrative purposes, the drawings show an embodiment which is presently preferred. However, it should be understood that the present disclosure is not limited to the embodiment illustrated in the drawings.

FIG. 1 illustrates an electrocautery system for transseptal access to a cardiac chamber of a patient, according to an embodiment of the present disclosure.

FIG. 2 illustrates a distal end segment and an intermediate segment of an electrocautery device of the electrocautery system of FIG. 1 in an axially elongated configuration.

FIG. 3 illustrates the distal end segment and the intermediate segment of the electrocautery device of FIG. 2 in an axially shortened configuration.

FIG. 4 illustrates a side sectional view of the distal end segment of the electrocautery device of FIG. 2.

FIG. 5 illustrates an electrocautery system for transseptal access to a cardiac chamber of a patient, according to another embodiment of the present disclosure.

FIG. 6 illustrates a flow chart of a method for transseptal access to the left atrium of a patient, according to an embodiment of the present disclosure.

FIG. 7 illustrates a distal tip portion of a catheter of an electrocautery system being positioned against the atrial septum of the patient, according to the method of FIG. 6.

FIG. 8 illustrates a distal end segment of an electrocautery device being advanced to the right atrial side of the atrial septum of the patient, according to the method of FIG. 6.

FIG. 9 illustrates a distal end segment and an intermediate segment of the electrocautery device, in an axially elongated configuration, being advanced from the right atrial side of the atrial septum to the left atrial side of the atrial septum and thereby forming a perforation through the atrial septum, according to the method of FIG. 6.

FIG. 10 illustrates the intermediate segment of the electrocautery device being reconfigured from the axially elongated configuration to an axially shortened configuration on the left atrial side of the atrial septum, according to the method of FIG. 6.

FIG. 11 illustrates the catheter being advanced through the perforation in the atrial septum to thereby dilate the perforation, according to the method of FIG. 6.

FIG. 12 illustrates a medical device delivery system being advanced through the catheter, the atrial septum, and into the left atrium, according to the method of FIG. 6.

### DETAILED DESCRIPTION OF THE DRAWINGS

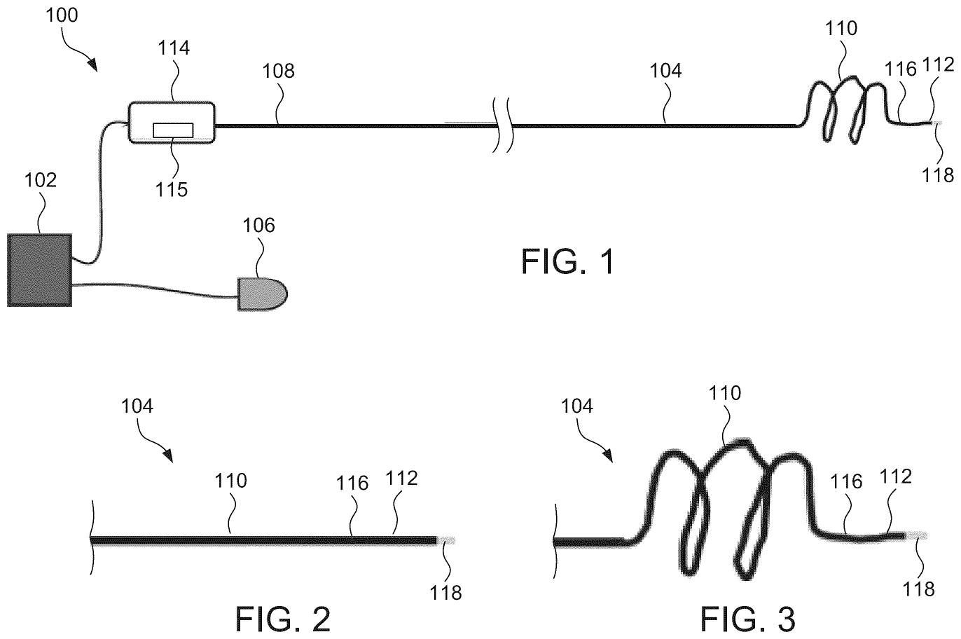

Referring to the drawings, wherein like reference numerals identify corresponding or similar elements throughout the several views, FIG. 1 illustrates an electrocautery system 100 for transseptal access to a cardiac chamber of a patient, according to an embodiment of the present disclosure. The electrocautery system 100 generally includes an energy source 102 that is coupled to and provides electrical energy to an elongated electrocautery device 104, which may also be referred to as a “wire” or “cable”. The electrocautery device 104 converts the electrical energy to thermal energy and delivers the thermal energy to a cardiac septum of a patient (for example, the atrial septum of the patient), and the device 104 thereby forms a perforation through the cardiac septum of the patient. The energy source 102 is also coupled to a user input 106 (for example, a foot pedal) that is operable to cause the energy source 102 to selectively deliver the electrical energy to the electrocautery device 104. These components and other features of the electrocautery system 100 are provided in the following paragraphs.

The energy source 102 may provide electrical energy having characteristics that are commonly associated with electrocautery procedures. For example, the energy source 102 may provide DC or AC electrical energy within a current range of 10 mA to 2 A, and/or a power range of 25-500 W. The electrocautery device 104 can be either monopolar or bipolar (that is, including two electrical subassemblies or wires running through the length of the device 104). The electrocautery device 104 may include a safety 115, such as an impedance or current-based safety, that automatically deenergizes the device 104 after forming a perforation through the cardiac septum of a patient.

With continued reference to FIG. 1 and additional reference to FIGS. 2 and 3, the electrocautery device 104 generally includes a proximal end segment 108, an intermediate segment 110 coupled to the proximal end segment 108, and a distal end segment 112 coupled to the intermediate segment 110 opposite the proximal end segment 108. The proximal end segment 108 remains outside of the patient during use, and the intermediate segment 110 and the distal end segment 112 are positionable within the patient. More specifically, the intermediate segment 110 is partially positionable in the heart of the patient and the distal end segment 112 is completely positionable within the patient. Generally, the proximal end segment 108 couples the device 104 to the energy source 102 via a coupler 114, and the distal end segment 112 delivers thermal energy to the cardiac septum of the patient and thereby forms a perforation through the cardiac septum.

The electrocautery device 104 may have dimensions appropriate for use with both adult and pediatric patients. For example, the electrocautery device 104 may have an overall length in a range of 30 cm to 200 cm, the intermediate segment 110 may have a length in a range of 0.5 cm to 6 cm, and the distal end segment 112 may have a length in a range of 5 mm to 10 mm. The electrocautery device 104 may have a diameter of about 3 French (3 Fr), or in a range of 0.5 mm to 1.25 mm.

With specific reference to FIGS. 2 and 3, the intermediate segment 110 of the electrocautery device 104 is reconfigurable from an axially elongated configuration (FIG. 2), for example, an initial configuration in which the distal end segment 112 perforates the cardiac septum, to an axially shortened configuration (FIG. 3), for example, a subsequent configuration after the distal end segment 112 perforates the cardiac septum. The intermediate segment 110 reconfigures to the axially shorted configuration to avoid inadvertent contact of the distal end segment 112 with other cardiac tissues, thereby inhibiting damage to such tissues. That is, the device 104 is designed to limit the extension of the distal end segment 112 into the heart after it pierces the cardiac septum. In some embodiments, the intermediate segment 110 is passively reconfigurable from the axially elongated configuration to the axially shortened configuration. For example, the intermediate segment 110 may be constructed from a shape memory material, set in the axially shortened configuration, and initially maintained in the axially elongated configuration by being received in a catheter (shown elsewhere). In other embodiments and as described in further detail below, the intermediate segment 110 is actively reconfigurable from the axially elongated configuration to the axially shortened configuration. In some embodiments, the intermediate segment 110 is in a range of 25 percent to 80 percent axially shorter in the axially shortened configuration than the axially elongated configuration. In some embodiments, the axially elongated configuration is a straight configuration, as illustrated, or a slightly coiled configuration. In some embodiments, the axially shortened configuration is a coiled configuration, as illustrated, or a further coiled configuration.

In certain embodiments, the distal end segment 112 of the electrocautery device 104 may be relatively stiff, to facilitate perforating the cardiac septum, compared to the intermediate segment 110 and/or the proximal end segment 108, to facilitate maneuvering through the vasculature of the patient. Stated another way, the distal end segment 112 has a first stiffness, the intermediate segment 110 has a second stiffness, and the second stiffness is less than the first stiffness. Such different stiffnesses may be provided by constructing the segments of the electrocautery device 104 with different structures, materials with different properties, and/or subjecting the segments to different treatments, such as heat treatments.

In certain embodiments, the coupler 114 is fixedly coupled to the proximal end segment 108 of the electrocautery device 104. In other embodiments, the coupler 114 is detachably coupled to the proximal end segment 108 of the electrocautery device 104 so the device 104 can be used in a similar manner as a standard guide wire for exchanging catheters.

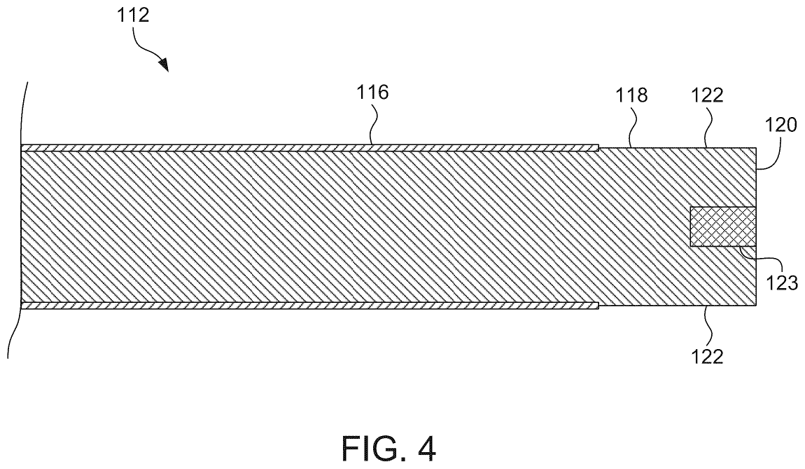

FIG. 4 illustrates a side section view of the distal end segment 112 of the electrocautery device 104. The distal end segment 112 includes a thermally insulating layer 116 and a thermally conductive tip 118 that is at least partially exposed from the thermally insulating layer 116. More specifically, the leading edge 120 and/or sections of the side surfaces 122 of the thermally conductive tip 118 are exposed from the thermally insulating layer 116. The thermally insulating layer 116 and the thermally conductive tip 118 together define a specific location from which the electrocautery device 104 delivers thermal energy to the tissue of the patient. The exposed portion of the thermally conductive tip 118 may have an axial length in a range of 0.5 mm to 5 mm. The thermally insulating layer 116 may be constructed of various appropriate materials, such as polymer composites, having insulating properties appropriate for the thermal energy expected. The thermally conductive tip 118 may be constructed of various appropriate materials, such as metals. The thermally conductive tip 118 may receive thermal energy dissipated by one or more electrical circuits (not shown) of the intermediate segment 110 of the electrocautery device 104. Relatedly, the thermally insulating layer 116 may extend into the intermediate segment 110 and surround such electrical circuits and other structures, such as a flexible tube, for example a laser-cut hypotube. The device 104 may include a pressure sensor 123 illustratively at the tip 118.

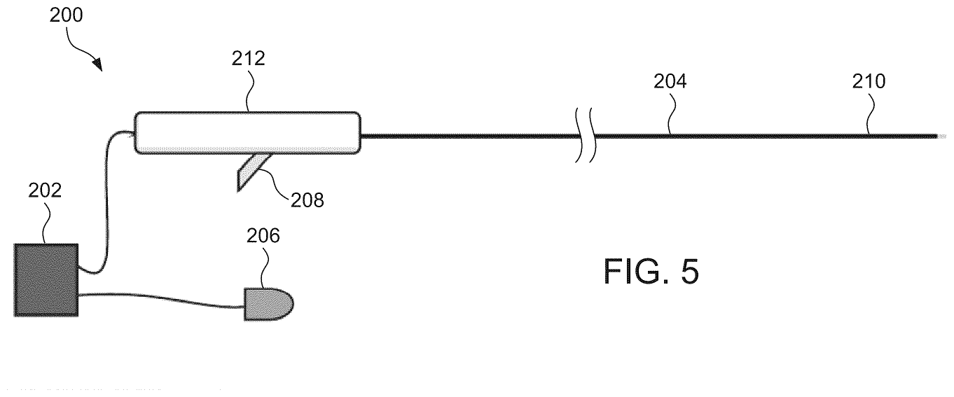

FIG. 5 illustrates an electrocautery system 200 for transseptal access to a cardiac chamber of a patient, according to another embodiment of the present disclosure. The electrocautery system 200 generally includes an energy source 202 that is coupled to and provides electrical energy to an elongated electrocautery device 204. The electrocautery device 204 converts the electrical energy to thermal energy and delivers the thermal energy to a cardiac septum of a patient (for example, the atrial septum of the patient), and the device 204 thereby forms a perforation through the cardiac septum of the patient. The energy source 202 is also coupled to a first user input 206 (for example, a foot pedal) that is operable to cause the energy source 202 to selectively deliver the electrical energy to the electrocautery device 204.

The electrocautery system 200 is generally similar to the system 100 described hereinabove. For example, the energy source 202 and the first user input 206 may be the same as or similar to the energy source 102 and the user input 106, respectively. The electrocautery device 204 is also generally similar to the electrocautery device 104, although the device 204 further includes a second user input 208 for actively controlling reconfiguration of the intermediate segment 210 from an axially elongated configuration (as illustrated in FIG. 5) to an axially shortened configuration (similar to the configuration of the device 104 illustrated in FIG. 3). For example, the second user input 208 may be operatively coupled to one or more pull wires (not shown) extending through the electrocautery device 204. The second user input 208 may be coupled to the coupler 212 of the device 204, as illustrated, or located elsewhere.

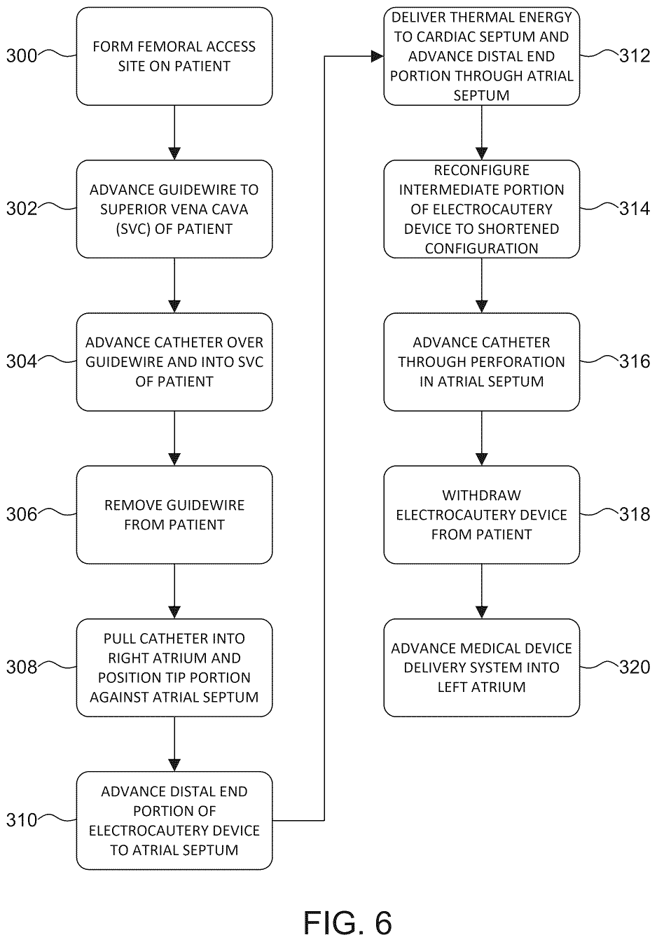

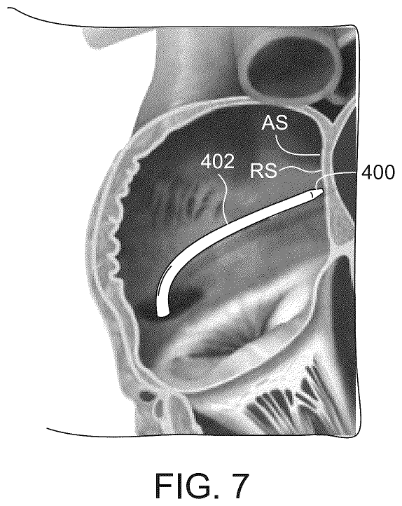

FIG. 6 illustrates a flow chart of a method for transseptal access to a cardiac chamber of a patient, according to an embodiment of the present disclosure. FIGS. 7-12 illustrate specific actions associated with the method. The method may incorporate medical imaging, such as intracardiac/transesophageal/transthoracic echocardiography and/or fluoroscopy, to facilitate device placement in the patient. The method begins at block 300 by forming an access site, more specifically a femoral access site, on the patient using a needle, such as an 18-gauge needle for an adult patient or a 21-gauge needle for a pediatric patient. Next, at block 302 a guidewire, such as J-tip guidewire, is advanced from the access site and into the vasculature of the patient. More specifically, a distal end segment of the guidewire is advanced into the superior vena cava (SVC) of the patient. At block 304, a catheter, such as a 8 Fr catheter for an adult patient or a 6 Fr catheter for a pediatric patient, is advanced from the access site, over the guidewire, and into the SVC of the patient. Next, at block 306 the guidewire is removed from the patient. At block 308, the catheter is pulled back into the cardiac chamber, more specifically the right atrium, and, as shown in FIG. 7, a distal tip portion 400 of the catheter 402 is positioned against a first side of the cardiac septum, more specifically the right atrial side RS of the atrial septum AS.

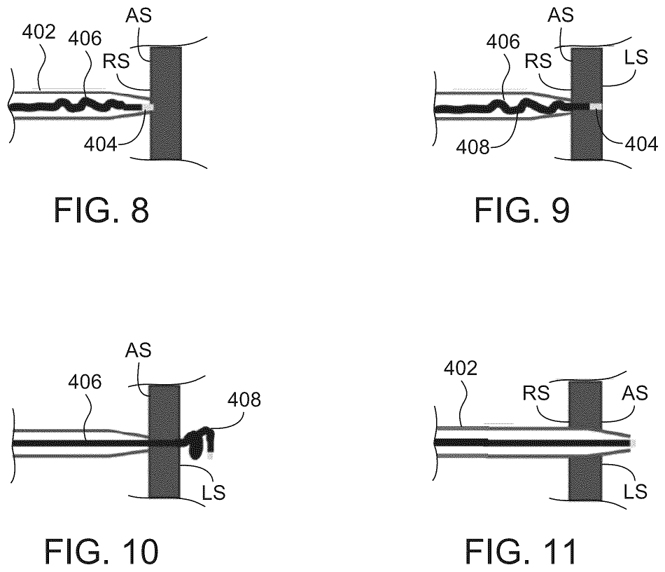

At block 310 and as shown in FIG. 8, the distal end segment 404 of an electrocautery device 406, which may be the electrocautery device 104 or the electrocautery device 204, is advanced from the access site, through the catheter 402, and to the first side of the cardiac septum, more specifically the right atrial side RS of the atrial septum AS. Next, at block 312 the electrocautery device 406 receives electrical energy from an energy source, such as the energy source 102, and the distal end segment 404 of the electrocautery device 406 delivers thermal energy to the cardiac septum, more specifically the atrial septum AS. Simultaneously and as shown in FIG. 9, the distal end segment 404 and an intermediate segment 408 of the electrocautery device 406, in an axially elongated configuration, are advanced from the first side of the cardiac septum to a second side of the cardiac septum, more specifically from the right atrial side RS of the atrial septum AS to the left atrial side LS of the atrial septum AS. The electrocautery device 406 thereby forms a perforation through the cardiac septum.

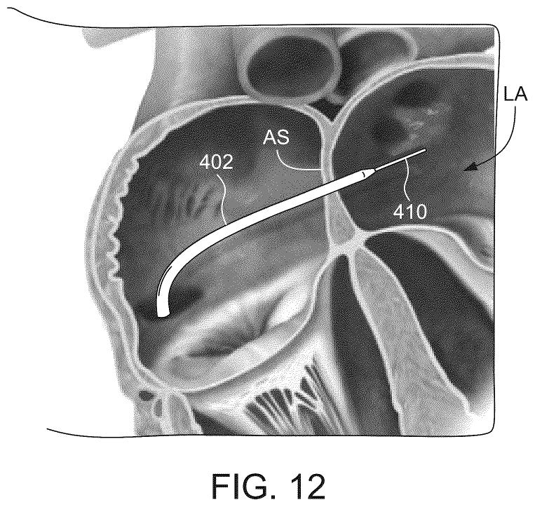

At block 314 and as shown in FIG. 10, the intermediate segment 408 of the electrocautery device 406 is then preferably reconfigured from the axially elongated configuration to an axially shortened configuration on the second side of the cardiac septum, more specifically on the left atrial side LS of the atrial septum AS. At block 316 and as shown in FIG. 11, the catheter 402 is then advanced through the perforation in the cardiac septum, more specifically from the right atrial side RS of the atrial septum AS to the left atrial side LS of the atrial septum AS, to dilate the perforation. At block 318, the electrocautery device 406 is withdrawn from the patient. At block 320 and as shown in FIG. 12, a medical device delivery system 410 (such as a transcatheter mitral valve repair (TMVr) system, a mitral valve in valve (VinV) implantation system, a transcatheter mitral valve replacement (TMVR) system, or the like) is advanced through the catheter 402, the cardiac septum, and into the contralateral cardiac chamber, more specifically through the atrial septum AS and into the left atrium LA. The medical device delivery system 410 may then be used according to known methods. Alternatively, instead of receiving the medical device delivery system 410, the catheter 402 may dilate the perforation to create a pressure-reducing shunt in the atrial septum AS.

Electrocautery systems, devices, and methods according to the present disclosure may provide one or more of the following advantages: increased success rates compared to methods that use needles and RF devices; improved value-based care and precision; increased speed compared to methods that use RF devices; reduced cardiac catheterization laboratory time, costs, and use of other resources, such as blood products; reduced risk of vascular complications; reduced exposure to radiation.

For the purposes of promoting an understanding of the principles of the invention, reference has been made to the preferred embodiments illustrated in the drawings, and specific language has been used to describe these embodiments. However, no limitation of the scope of the invention is intended by this specific language, and the invention should be construed to encompass all embodiments that would normally occur to one of ordinary skill in the art.

The particular implementations shown and described herein are illustrative examples of the invention and are not intended to otherwise limit the scope of the invention in any way. The use of any and all examples, or exemplary language (for example, “such as”) provided herein, is intended merely to better illuminate the invention and does not pose a limitation on the scope of the invention unless otherwise claimed. Numerous modifications and adaptations will be readily apparent to those skilled in this art without departing from the spirit and scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (that is, meaning “including, but not limited to,”) unless otherwise noted. The term “connected” is to be construed as partly or wholly contained within, attached to, or joined together, even if there is something intervening.

The recitation of ranges of values herein are merely intended to serve as a shorthand method of referring individually to each separate value falling within the range, unless otherwise indicated herein, and each separate value is incorporated into the specification as if it were individually recited herein.

Terms such as “about” or “approximately”, unless otherwise defined or restricted in the specification, should be understood to define a variance of plus or minus 5 percent to 10 percent to the numerical term referred to.

All methods described herein can be performed in any suitable order unless otherwise indicated herein or otherwise clearly contradicted by context. The use of any and all examples, or exemplary language (for example, “such as”) provided herein, is intended merely to better illuminate embodiments of the invention and does not impose a limitation on the scope of the invention unless otherwise claimed. The various embodiments and elements can be interchanged or combined in any suitable manner as necessary.

The use of directions, such as forward, rearward, top and bottom, upper and lower are with reference to the embodiments shown in the drawings and, thus, should not be taken as restrictive. Reversing or flipping the embodiments in the drawings would, of course, result in consistent reversal or flipping of the terminology.

No language in the specification should be construed as indicating any non-claimed element as essential to the practice of the invention.

It will be apparent to those skilled in the art that various modifications and variations can be made to the present invention without departing from the spirit and scope of the invention. There is no intention to limit the invention to the specific form or forms disclosed, but on the contrary, the intention is to cover all modifications, alternative constructions, and equivalents falling within the spirit and scope of the invention, as defined in the appended claims. Thus, it is intended that the present invention cover the modifications and variations of this invention provided they come within the scope of the appended claims and their equivalents.

What is claimed is:

1. An electrocautery device for transseptal access to a cardiac chamber of a patient, comprising:
a proximal end segment configured to remain external from the patient;
an intermediate segment coupled to the proximal end segment, the intermediate segment configured to be at least partially disposed in the heart of the patient, a portion of the intermediate segment being reconfigurable from an axially elongated configuration to an axially shortened configuration; and
a distal end segment coupled to the intermediate segment opposite the proximal end segment, the distal end segment configured to be disposed in the heart of the patient, and the distal end segment configured to deliver thermal energy to a cardiac septum of the heart of the patient and form a perforation through the cardiac septum.

2. The electrocautery device of claim 1, wherein the axially shortened configuration is a coiled configuration.

3. The electrocautery device of claim 1, wherein the distal end segment has a first stiffness, the intermediate segment has a second stiffness, and the second stiffness is less than the first stiffness.

4. The electrocautery device of claim 1, wherein the distal end segment includes:
a thermally insulating layer; and
a thermally conductive tip at least partially exposed from the thermally insulating layer to facilitate delivering the thermal energy to the cardiac septum of the heart of the patient.

5. The electrocautery device of claim 4, wherein the thermally insulating layer extends into the intermediate segment.

6. The electrocautery device of claim 1, wherein the intermediate segment is passively reconfigurable from the axially elongated configuration to the axially shortened configuration.

7. The electrocautery device of claim 1, wherein the intermediate segment is actively reconfigurable from the axially elongated configuration to the axially shortened configuration.

8. An electrocautery system for transseptal access to a cardiac chamber of a patient, comprising:
an energy source configured to provide electrical energy;
an electrocautery device configured to receive the electrical energy from the energy source and convert the electrical energy to thermal energy, the electrocautery device comprising:
a proximal end segment configured to remain external from the patient;
an intermediate segment coupled to the proximal end segment, the intermediate segment configured to be at least partially disposed in the heart of the patient, a portion of the intermediate segment being reconfigurable from an axially elongated configuration to an axially shortened configuration; and
a distal end segment coupled to the intermediate segment opposite the proximal end segment, the distal end segment configured to be disposed in the heart of the patient, and the distal end segment configured to deliver the thermal energy to a cardiac septum of the heart of the patient and form a perforation through the cardiac septum.

9. The electrocautery system of claim 8, wherein the distal end segment has a first stiffness, the intermediate segment has a second stiffness, and the second stiffness is less than the first stiffness.

10. The electrocautery system of claim 8, wherein the distal end segment of the electrocautery device includes:
a thermally insulating layer; and
a thermally conductive tip at least partially exposed from the thermally insulating layer to facilitate delivering the thermal energy to the cardiac septum of the heart of the patient.

11. The electrocautery system of claim 10, wherein the thermally insulating layer extends into the intermediate segment.

12. The electrocautery system of claim 8, wherein the axially shortened configuration is a coiled configuration.

13. The electrocautery system of claim 8, further comprising a catheter configured to receive the electrocautery device.

14. A method for transseptal access to a cardiac chamber of a patient, comprising the steps of:
advancing a distal end segment of an electrocautery device from an access site on the patient to a first side of a cardiac septum in the heart of the patient;
delivering thermal energy from the distal end segment of the electrocautery device to the cardiac septum;
advancing the distal end segment and a portion of an intermediate segment of the electrocautery device, in an axially elongated configuration, from the first side of the cardiac septum to a second side of the cardiac septum, the electrocautery device thereby forming a perforation through the cardiac septum; and
reconfiguring the portion of the intermediate segment of the electrocautery device from the axially elongated configuration to an axially shortened configuration on the second side of the cardiac septum.

15. The method of claim 14, wherein the step of advancing the distal end segment of the electrocautery device from the access site to the first side of the cardiac septum comprises advancing the electrocautery device within a catheter.

16. The method of claim 14, further comprising the steps of, after reconfiguring the portion of the intermediate segment of the electrocautery device to the axially shortened configuration, advancing a catheter through the perforation in the cardiac septum to thereby dilate the perforation in the cardiac septum.

17. The method of claim 16, further comprising the steps of:
after reconfiguring the portion of the intermediate segment of the electrocautery device to the axially shortened configuration, withdrawing the electrocautery device from the patient; and
after withdrawing the electrocautery device from the patient, advancing a medical device delivery system through the catheter.

18. The method of claim 14, wherein the axially shortened configuration is a coiled configuration.

19. The method of claim 14, wherein the cardiac septum is an atrial septum.

20. The method of claim 14, wherein the access site is a femoral access site.
